(19) United States
(12) Patent Application Publication
(10) Pub. No.: US 2023/0295065 A1
(43) Pub. Date: Sep. 21, 2023

(54) System for Ultrapurification of Organic Solvent

(71) Applicant: New Jersey Institute of Technology, Newark, NJ (US)

(72) Inventors: Kamalesh Sirkar, Bridgewater, NJ (US); John Chau, Belleville, NJ (US)

(73) Assignee: New Jersey Institute of Technology, Newark, NJ (US)

(21) Appl. No.: 18/118,547

(22) Filed: Mar. 7, 2023

Related U.S. Application Data
(60) Provisional application No. 63/320,445, filed on Mar. 16, 2022.

(51) Int. Cl. C07C 29/80; B01D 61/36

(52) U.S. Cl. CPC C07C 29/80; B01D 61/364; B01D 61/366; B01D 2325/02832

(57) ABSTRACT

A method of purifying an organic solvent is provided. The method includes introducing an organic solvent into a membrane distillation system. The membrane distillation system includes a perfluorodioxole membrane. The method includes performing a distillation technique with the membrane distillation system using the perfluorodioxole membrane to treat and purify the organic solvent.

### CROSS-REFERENCE TO RELATED APPLICATIONS

This application claims the benefit of co-pending U.S. Provisional Patent Application No. 63/320,445, which was filed on Mar. 16, 2022. The entire content of the foregoing provisional patent application is incorporated herein by reference.

### FIELD OF THE DISCLOSURE

The present disclosure relates to a system and method for vacuum membrane distillation. In particular, the present disclosure relates to a system including a membrane for ultrapurification of organic solvents, such as isopropyl alcohol, by vacuum membrane distillation.

### BACKGROUND

Silicon wafers used in semiconductor manufacturing undergo extraordinary cleaning to ensure that there are no residues of any kind left on the wafer that can introduce defects in chips. Therefore, the water used for the cleaning process is ultrapure water (UPW) having extremely high purity, e.g., metals at parts per trillion level. At a particular cleaning stage, organic solvent-based cleaning is undertaken to remove from the surface of silicon wafers any oils and organic residues. During this cleaning step, solvents used should not introduce any further contaminant on the wafer. A common organic solvent used is isopropyl alcohol (IPA). In general, ultrapure IPA is needed, which may be referred to herein and in the industry as “electronic grade IPA”.

The impurities in IPA can be nonvolatile, semi-volatile, or volatile. To reach an acceptable level for cleaning wafers, it is necessary to remove all nonvolatile contaminants from the IPA. In general, trace volatile contaminants will disappear from the wafer surface soon after exposure. Semi-volatile impurities typically disappear after a certain period of time.

Bulk IPA from the marketplace generally has significant levels of impurity of various kinds. Evaporation of such IPA and its condensation could remove nonvolatile impurities. However, there may be residual amounts of volatile impurities left in the condensed IPA. Lower temperature IPA evaporation operation coupled with vacuum and subsequent condensation can achieve further purification vis-à-vis semi-volatile impurities. One such evaporation technique is Vacuum Membrane Distillation (VMD), which has been employed in desalination (See, e.g., Li, B. et al., Novel Membrane and Device for Vacuum Membrane Distillation-based Desalination Process, J. Membrane Sci., 257 (1-2), p. 60-75 (2005)). Another evaporation technique uses an inert sweep gas (out of necessity), instead of a vacuum, and a condenser to condense the volatilized IPA. See id.

In membrane distillation, the nonvolatile components of a solution can be retained in the feed solution by a porous hydrophobic membrane, while the volatile parts can be transported through the porous membrane. The vapor transfer can be caused by a vapor pressure difference induced by a temperature difference across the membrane. This enables the separation of nonvolatile components from a solution.

In VMD, liquid IPA feed flows on one side of the porous hydrophobic membrane, which is subjected to vacuum on the other side of the membrane. The membrane pores should be gas-filled and not be wetted by IPA. The permeated vapor coming out of the permeate side of the membrane is taken to a condenser. In the laboratory, the condenser could be cooled by dry ice/liquid N₂. Gas chromatography/mass spectrometry (GCMS) testing of the condensate vis-à-vis the feed IPA could indicate the purity of the evaporated IPA.

There is an unmet need for an improved membrane distillation process that yields ultrapure organic solvents using improved membranes.

### SUMMARY

In accordance with embodiments of the present disclosure, exemplary embodiments are directed to a perfluorodioxole copolymer. In one or more embodiments, a perfluoropolymer, perfluoro-2,2-dimethyl-1,3-dioxole copolymerized with tetrafluoroethylene, (PDD-TFE), designated CMS-7, is disclosed. This amorphous glassy extremely hydrophobic copolymer has a very high free volume (FV) fraction. The maximum dimension (radius) of FV regions is less than about 0.65 nm, allowing a single solvent molecule permeation. Further, interactions between polarity, dimensions, and shapes of solvent molecules with those of polymer FV elements can lead to extraordinarily selective permeation.

Because IPA has a low surface tension of 23 dyne/cm at 20° C., IPA will wet most porous hydrophobic membranes. A superhydrophobic membrane surface coupled with a smaller pore size of the membrane and aided by not too high a vacuum (e.g., a vacuum level of less than full vacuum (29.92 in Hg) on the permeate side is likely to prevent IPA leakage to the permeate side provided the critical surface tension of the membrane material is significantly lower than the surface tension of IPA. (See, e.g., Li, L. et al., Studies in Vacuum Membrane Distillation with Flat Membranes, J. Membrane Sci., 523, p. 225-234 (2017)). Accordingly, porous/microporous fluoropolymer surfaces could be used. This will also ensure a high enough flux of IPA vapor through the microporous membrane. Purification data from such a hydrophobic/superhydrophobic membrane is discussed herein.

In one embodiment, a membrane with a pore size from about 1 nanometer to about a sub-nanometer level using a fluoropolymer can be used such that pore wetting issues can be bypassed if vacuum is used to drive vaporized IPA. A full vacuum level can be used on the permeate side. In some embodiments, the flux of IPA is substantially enhanced by raising the temperature of the feed IPA being processed at the cost of enhancing the flux of volatile contaminants in feed IPA. Volatile and semi-volatile species may still be transmitted to some extent through such a membrane having about 1 nm to about a sub-nanometer membrane.

To reduce the transmission of volatile and semi-volatile species, the subnanopore dimension (i.e., radius) can be reduced to the level of about, e.g., 0.5-0.8 nm inclusive, 0.5-0.7 nm inclusive, 0.5-0.6 nm inclusive, 0.6-0.8 nm inclusive, 0.7-0.8 nm inclusive, 0.5 nm, 0.6 nm, 0.7 nm, 0.8 nm, or the like, in radius and employ glassy perfluorinated/perfluoropolymer membranes in one or more embodiments, which swell very little in organic solvents and allow primarily smaller volatile species to go through their free volume regions of larger dimensions. There is very little, if any, transmission via conventional solution and diffusion through the smaller free volume regions of such polymers. Larger molecules are likely to be conveniently excluded or significantly hampered in going through such membranes. Such evaporative purification of IPA can produce a much purer IPA than conventional distillation or other processes. In addition, it has been postulated that alcohols undergo dimerization to a significant extent in contact with such types of polymers, and potentially completely occupy the large dimensions of the free volume regions of these polymers and exclude many other compounds. (See, e.g., Chau, J. et al., Organic Solvent Mixture Separation during Reverse Osmosis and Nanofiltration by a Perfluorodioxole Copolymer Membrane, J. Membrane Science, 618, 118663 (2021)).

In accordance with embodiments of the present disclosure, an exemplary method of purifying an organic solvent is provided. The method includes introducing an organic solvent into a membrane distillation system. The membrane distillation system includes a perfluorodioxole membrane. The method includes performing a distillation technique with the membrane distillation system using the perfluorodioxole membrane to treat and purify the organic solvent.

In some embodiments, the perfluorodioxole membrane can have a dioxole content of about 65% to about 87%, inclusive. In some embodiments, the perfluorodioxole membrane includes a perfluoropolymer of perfluoro-2,2-dimethyl-1,3-dioxole copolymerized with tetrafluoroethylene membrane. In some embodiments, the perfluorodioxole membrane includes a HYFLON® AD perfluoropolymer or a CYTOP® AD perfluoropolymer.

In some embodiments, the distillation technique includes a vacuum membrane distillation process. In some embodiments, the distillation technique includes a sweep gas membrane distillation process. In some embodiments, the distillation technique includes an air gap membrane distillation process. In some embodiments, the distillation technique includes a direct contact membrane distillation process.

In some embodiments, the organic solvent can be isopropyl alcohol. In some embodiments, the organic solvent can be isopropyl alcohol, toluene, ethanol, methanol, or butanol. Treating and purifying the organic solvent results in the organic solvent being free from all non-volatile contaminants. In some embodiments, the perfluorodioxole membrane can be a perfluorodioxole copolymer membrane. In some embodiments, the perfluorodioxole membrane includes a pore size between about 1 nanometer and about a sub-nanometer.

In accordance with embodiments of the present disclosure, an exemplary system for purifying an organic solvent is provided. The system includes a feed reservoir including an organic solvent, and a perfluorodioxole membrane fluidically connected to the feed reservoir. The system includes a pump configured to pump the organic solvent from the feed reservoir to and over the perfluorodioxole membrane. Passage of some of the organic solvent through the perfluorodioxole membrane performs a distillation technique to treat and purify the organic solvent.

The perfluorodioxole membrane can include a first side exposed to the organic solvent pumped to the perfluorodioxole membrane by the pump. The perfluorodioxole membrane includes a second side opposing the first side, and the second side is exposed to one or more vacuum levels. The system can include a vacuum pump fluidically connected to the second side of the perfluorodioxole membrane to expose the second side to the one or more vacuum levels. The system can include a cold trap fluidically connected to the second side of the perfluorodioxole membrane to collect condensate from the perfluorodioxole membrane. The system can include a temperature probe capable of measuring a temperature of the organic solvent prior to entry of the organic solvent into the device having the perfluorodioxole membrane.

The perfluorodioxole membrane can include a perfluoropolymer of perfluoro-2,2-dimethyl-1,3-dioxole copolymerized with tetrafluoroethylene membrane. In some embodiments, the distillation technique can include at least one of a vacuum membrane distillation process, a sweep gas membrane distillation process, an air gap membrane distillation process, or a direct contact membrane distillation process. In some embodiments, the organic solvent can be isopropyl alcohol, toluene, ethanol, methanol, or butanol. Treating and purifying the organic solvent results in the organic solvent being free from all non-volatile contaminants.

Any combination and/or permutation of the embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the disclosed perfluorodioxole copolymer membrane and associated systems and methods, reference is made to the accompanying figures, wherein:

FIG. 1 shows a diagrammatic view of a system for vacuum membrane distillation in accordance with embodiments of the present disclosure;

FIG. 2 is a chromatogram for IPA samples, an extracted ion chromatogram (EIC) for hydrocarbon (HC), and direct injection;

FIG. 3 is a table of quantitative linear HCs in IPA samples in both feed and condensate for a CMS-7 membrane;

FIG. 4 is a chromatogram for IPA samples, tentatively identified compounds (TIC), and direct injection;

FIG. 5 is a graphical depiction showing a percent reduction of hydrocarbons after vacuum membrane distillation (VMD) for a 1 nm superhydrophobic membrane;

FIG. 6 shows a perfluoropolymer of the type perfluoro-2,2-dimethyl-1,3-dioxole copolymerized with tetrafluoroethylene, (PDD-TFE) that can be used in the present disclosure;

FIG. 7 shows a HYFLON® AD perfluoropolymer that can be used in the present disclosure; and

FIG. 8 shows a CYTOP® AD perfluoropolymer that can be used in the present disclosure.

### DETAILED DESCRIPTION

The present disclosure now will be described more fully hereinafter with reference to the accompanying drawings, in which illustrative embodiments of the invention are shown. In the drawings, the relative sizes of regions or features may be exaggerated for clarity. This present disclosure may, however, be embodied in many different forms and should not be construed as limited to the embodiments set forth herein; rather, these embodiments are provided so that this disclosure will be thorough and complete, and will fully convey the scope of the invention to those skilled in the art. Well-known functions or constructions may not be described in detail for brevity and/or clarity.

The terminology used herein is to describe particular embodiments only and is not intended to limit the scope of the invention. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Exemplary embodiments are directed to a perfluorodioxole copolymer membrane, and a system and/or method for ultrapurification of an organic solvent using a perfluorodioxole copolymer membrane. It should be understood that embodiments can generally be applied to other membranes. Although discussed herein with respect to vacuum membrane distillation, it should be understood that embodiments could generally be applied to other membrane distillation processes, e.g., sweep gas membrane distillation, air gap membrane distillation, direct contact membrane distillation (DCMD), or the like. In sweep gas membrane distillation, an inert gas (e.g., N₂) can be used to sweep away the IPA vapor to a condenser to condense the volatilized IPA. In air gap membrane distillation, the IPA vapor can be condensed by contacting a cold surface placed next to the perfluoropolymer membrane. In DCMD, cold ultrapure IPA liquid flowing on the other side can be used to condense the purified IPA in vapor form. Further, if there is no wetting of the pores by the IPA, then any of the membrane distillation processes discussed herein can be used. Likewise, although discussed herein with respect to isopropyl alcohol, it should be understood that embodiments could generally be applied to other organic solvents.

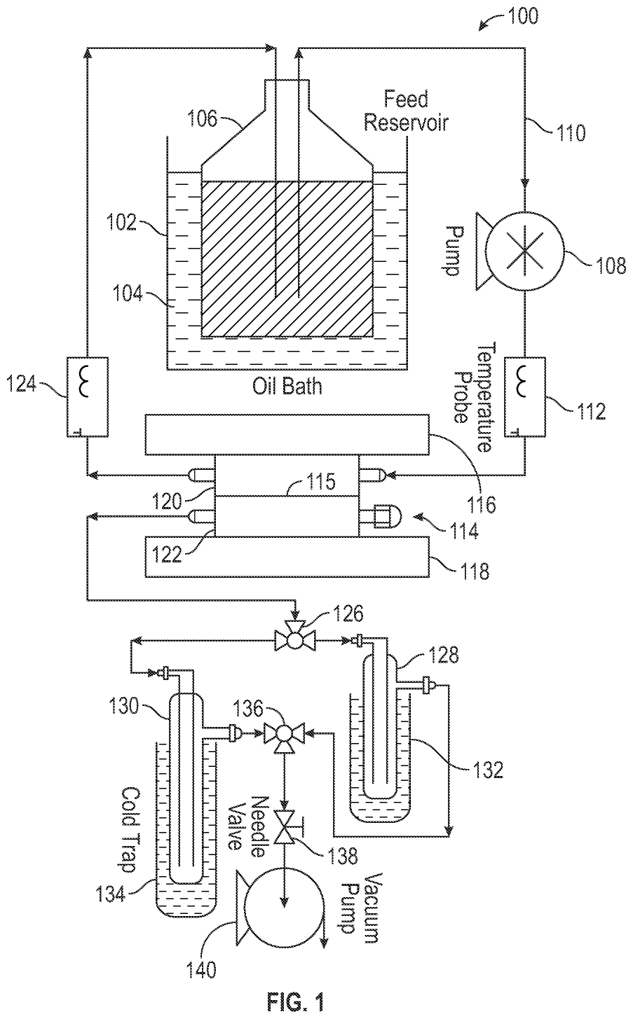

FIG. 1 is a diagrammatic view of a system 100 for vacuum membrane distillation in accordance with the present disclosure. The system 100 includes a tank 102 with an oil bath 104, and a feed reservoir 106 disposed at least partially within the oil bath 104. Downstream of the feed reservoir 106, the system 100 includes a pump 108 fluidically connected to the feed reservoir 106 via piping 110. The system 100 includes a temperature probe 112 disposed downstream of the pump 108 and fluidically connected to the pump 108 via piping 110, and top half 120 of a membrane distillation cell 114 fluidically connected to the output of the pump 108 going through piping 110 and the temperature probe 112. The membrane 115 in the membrane distillation test cell 114 is located between two halves (top half 120 and bottom half 122) of the membrane distillation test cell 114, and prevents fluid communication between these two halves of the test cell 114 via O-rings and gaskets, except for the contents that pass through the membrane 115 from the hot feed liquid side half 120 to the vacuum side half 122. The two halves 120, 122 of the test cell 114 are each supported and secured in place by two test cell cover plates 116, 118, respectively.

One hot liquid output line from the top half 120 of the membrane distillation test cell 114 connects to another temperature probe 124 of the system 100, and returns to the oil bath 104. A second output line from the bottom half 122 of the membrane distillation test cell 114 fluidically connects to a three-way valve 126, with one output connecting to a first cold trap 128 and a second output connecting to a second cold trap 130. Each cold trap 128, 130 can include a respective fluid tank 132, 134 filled with liquid N₂. The output of both cold traps 128, 130 fluidically connects to a three-way valve 136, which directs fluid to a needle valve 138. The needle valve 138 is, in turn, fluidically connected to a vacuum pump 140. A digital vacuum regulator can be used to regulate operation of the vacuum pump 140 in terms of the vacuum level needed to be maintained.

Different types of membranes 115 can be used in the system 100. In some embodiments, a brine feed can be located in the feed reservoir 106 (e.g., a vessel) in the oil bath 104. For example, IPA feed can be used in the feed reservoir 106 located in the oil bath 104. The feed can be pumped by pump 108 to one side of the membrane 115. The opposite side of the membrane 115 can be exposed to several vacuum levels by a vacuum pump 140 having a digital vacuum regulator. In some embodiments, the vacuum levels can be, e.g., 5 in Hg, 10 in Hg, 28 in Hg, 29 in Hg, 5-29 in Hg inclusive, 5-28 in Hg inclusive, 5-25 in Hg inclusive, 5-20 in Hg inclusive, 5-15 in Hg inclusive, 5-10 in Hg inclusive, 10-29 in Hg inclusive, 15-29 in Hg inclusive, 20-29 in Hg inclusive, 25-29 in Hg inclusive, 28-29 in Hg inclusive, 10-28 in Hg inclusive, or the like. The condensate can be collected in one or more glass vacuum traps 128, 130.

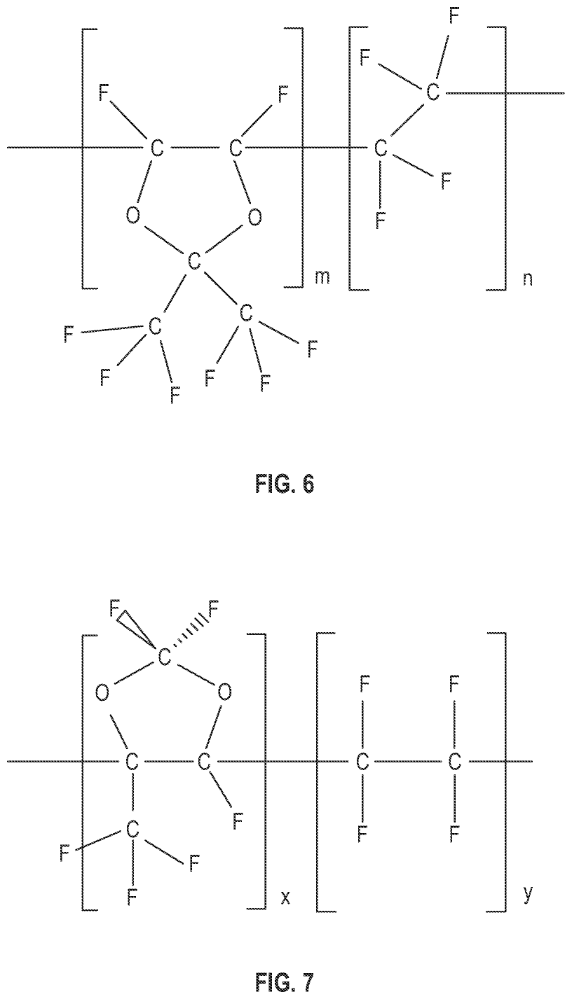

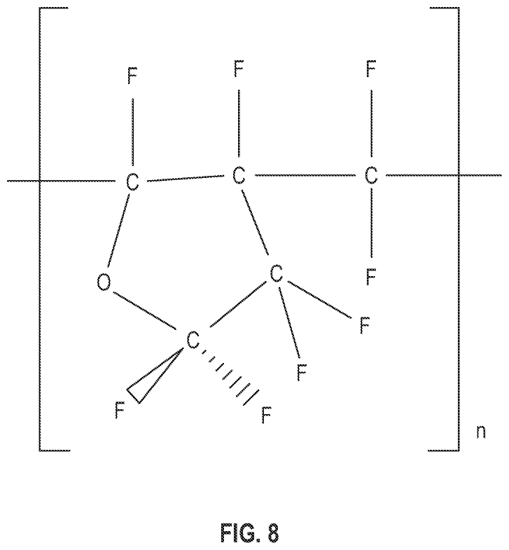

In some embodiments, the membrane 114 can be a perfluoropolymer, perfluoro-2,2-dimethyl-1,3-dioxole copolymerized with tetrafluoroethylene, (PDD-TFE). For example, FIG. 6 shows a perfluoropolymer of the type perfluoro-2,2-dimethyl-1,3-dioxole copolymerized with tetrafluoroethylene, (PDD-TFE) that can be used in the system 100. Two specific varieties of this copolymer, CMS-3 or CMS-7 (obtained under license from Chemours Inc.) from Compact Membrane Systems Inc., are available and have very high solvent resistance. Other membranes that can be used in the system 100 include, e.g., perfluoropolymers AF 1600 and AF 2400 (originally developed by DuPont and now manufactured by Chemours Inc.), or the like. AF 1600 has a dioxole content at about 65% and AF 2400 has a dioxole content at about 87%. The AF 1600 PDD-TFE copolymer may be essentially the same as CMS-3; and similarly, the AF 2400 PDD-TFE copolymer may be essentially identical to CMS-7. The values of m (=1−n) for the variety of copolymers listed herein are CMS-3, AF 1600, 65%; CMS-7, AF 2400, 87%. In some embodiments, any copolymer or PDD-TFE having dioxole content of, e.g., about 80%, between 65-87% inclusive, or the like, could be used as the membrane. In some embodiments, other perfluoropolymers, e.g., HYFLON® AD (FIG. 7), CYTOP® AD (FIG. 8), or the like, can be used as the membrane.

The materials and the methods of the present disclosure are described below. Although the discussion involves the use of specific materials, such as specific membranes, it should be understood that the exemplary system and the associated methods could be used with other suitable materials. Similar quantities or measurements may be substituted without altering the method embodied below.

The membrane used in the experimentation discussed herein is CMS-7, a perfluoropolymer material essentially identical to the polymer AF-2400. These polymers belong to the type, perfluoro-2,2-dimethyl-1,3-dioxole copolymerized with tetrafluoroethylene, (PDD-TFE).

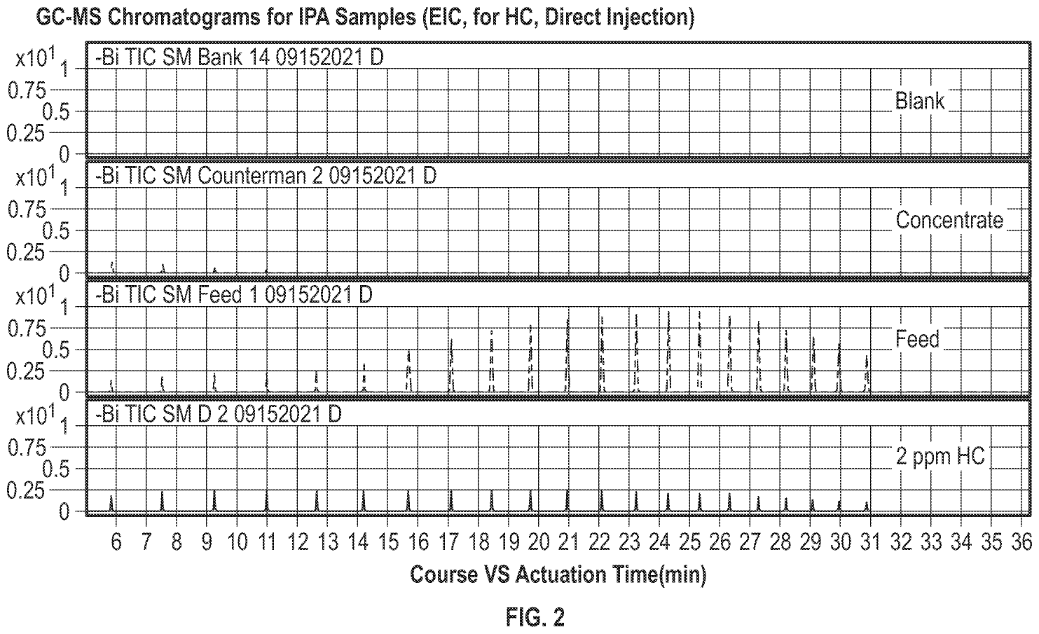

One example used for testing of the exemplary method includes the separation/purification performance of a flat piece of CMS-7 membrane exposed to hot IPA feed at 45° C. The downstream of such a membrane was exposed to a vacuum level of about 28 inch Hg plus. The dense polymer film thickness used was about 0.67 μm. This thickness can be reduced to as low as about 20 nm. The polymer film was supported on a porous expanded polytetrafluoroethylene (e-PTFE) support. The feed IPA flow rate was about 50 cc/min and the feed temperature was about 45° C. Full vacuum was applied in a vacuum membrane distillation system 100 as shown in FIG. 1. Since there was no water present with the IPA being purified, there was no need for the condenser arrangement. (See, e.g., FIG. 2B of L. Li et al., Studies in Vacuum Membrane Distillation with Flat Membranes, J. Membrane Sci., 523, 225-234 (2017)).

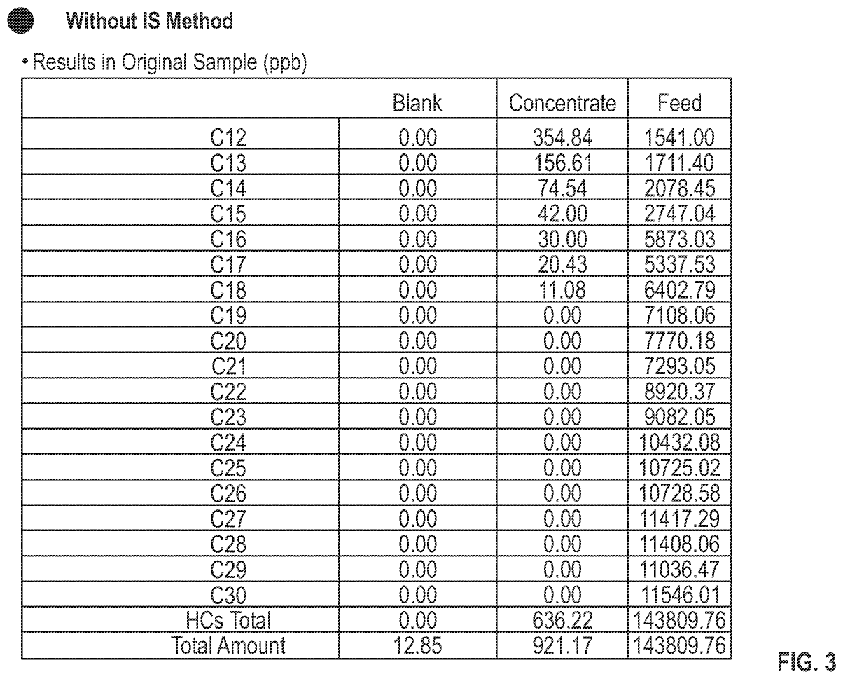

To test the purification capability of the membrane, the feed IPA was spiked with a 2 ppm spike (C7-C30 in hexane) in an IPA solution. The gas chromatography/mass spectrometry (GC-MS) results are shown in the chart of FIG. 2 and the table of FIG. 3. The top section of FIG. 2 shows data for a blank run with no sample. The second section from the top identifies only 3-4 tiny peaks in the IPA condensate (identified in FIG. 2 as the concentrate, and which is actually the IPA condensate). The third section from the top identifies the significant peaks of C7-C30 compounds introduced into the IPA feed by the 2 ppm spike added to the feed IPA sample. The fourth section from the top analyses the 2 ppm spike (C7-C30 in hexane) introduced in the feed. The second section identifying the composition of the condensate shows that all compounds higher than C11 compound are not visible, highlighting the extraordinary and unexpected purification achieved. Further, the concentration of the compounds lower than C11 are considerably smaller than their concentrations in the feed. This exemplifies the remarkable and unexpected level of purification achieved.

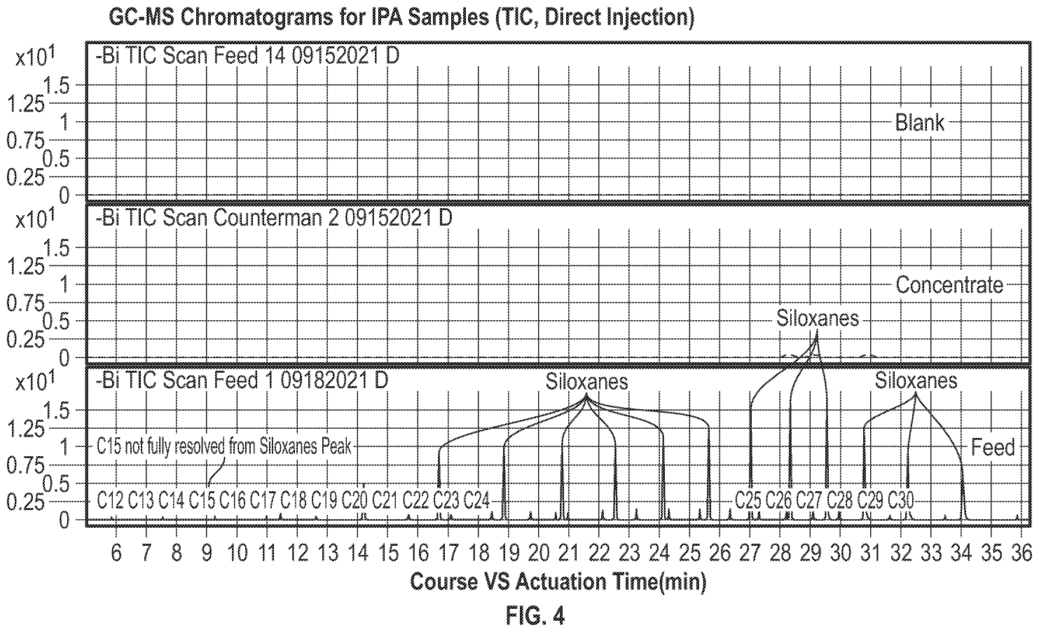

FIG. 3 provides the actual numerical values of the concentrations of the hydrocarbons in various samples. The condensate has very low concentration levels of C12 to C18 compared to those in the feed; C19-C30 compounds are absent in the condensate. This represents an extraordinary level of purification. The results were analyzed at Entegris Inc. These results indicate that the IPA condensate has undergone an exceptional purification to the extent of 99.36% of the original impurities being removed; further, trace levels of impurities present are primarily of lower molecular weight that are semivolatile. FIG. 4 is a chromatogram for IPA samples, tentatively identified compounds (TIC), and direct injection. It is noted from FIG. 4 that there may be siloxane in the feed that came from the column. Accordingly, the siloxane results can be ignored and the focus should be on the hydrocarbons. This result appears as FIG. 4, which has tentatively identified compounds (TIC).

The flux of IPA under these conditions was measured to be about 2.29 kg/m²-hr. The VMD experiment for CMS 7 was used in a cross-flow cell (the cell includes top and bottom halves 120, 122, respectively with a membrane 115 sandwiched in between). Both top and bottom halves 120, 122 have two openings.

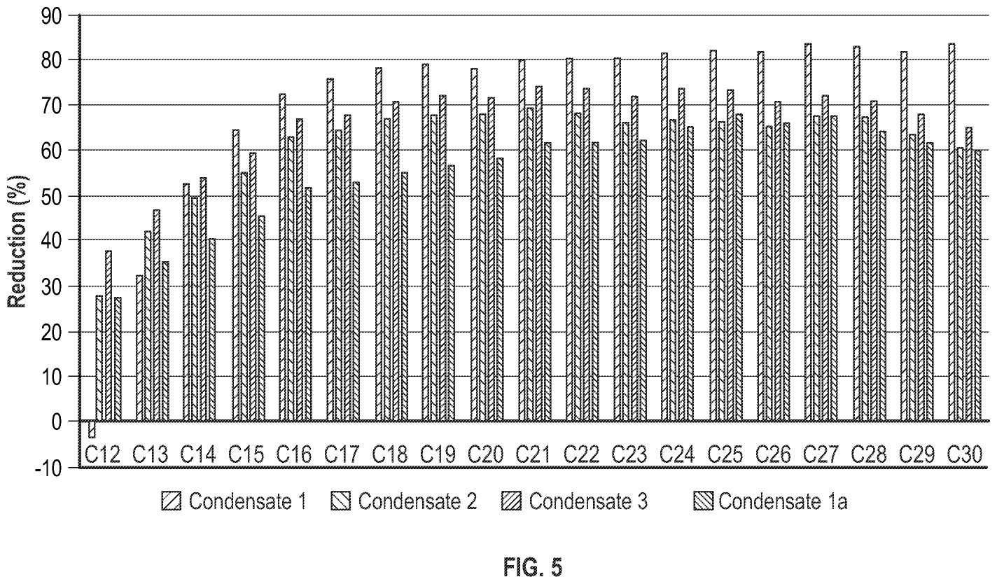

The table of FIG. 3 shows that out of 143,809 ppb of C7-C30 impurities injected into the feed, only 921 ppb show up in the condensate (the so-called concentrate). Further, there are no C19-C30 present in the permeate/condensate at all. In addition, the results of FIGS. 2 and 3 are to be compared with the results obtained during VMD with a 1 nm superhydrophobic membrane from Entegris Inc., which are shown in FIG. 5.

FIG. 5 shows the percent reductions of various hydrocarbons C13 to C30 in the condensate from a VMD process conducted using the apparatus of FIG. 1 with a 1 nm superhydrophobic membrane and the same feed spike in IPA as mentioned earlier. FIG. 5 shows that all such hydrocarbon species are present in significant amounts in the condensate even though their concentrations are reduced by anywhere from 30% to 80% of their amount in the feed. Further, the procedure and the feed spike samples were identical to that practiced with the CMS-7 perfluoropolymer membrane. In particular, feed IPA was spiked with a 2 ppm spike (C7-C30 in hexane) in IPA solution. The flux of IPA obtained with this 1 nm superhydrophobic membrane at 45° C. under a vacuum of 5 in Hg in the cross-flow cell at a feed flow rate of 50 cc/min used earlier was 1.13 kg/m²-hr.

There is another advantage of using the type of hydrophobic/superhydrophobic membrane being used via CMS-7, AF 2400, or the like. These materials are extremely hydrophobic and the liquid entry pressure (LEP) value is extraordinarily high compared to 7-10 psig for the 1 nm superhydrophobic membrane from Entegris Inc. The critical surface tension of extremely hydrophobic materials, such as TEFLON® is about 18 dyne/cm. The LEP value can be as high as 100 psig for IPA for CMS-7. (See, e.g., FIG. 3 of J. Chau et al., “Performance of a Composite Membrane of a Perfluorodioxole Copolymer in Organic Solvent Nanofiltration”, Separation and Purification Technology, 199, 233-241 (2018)). Flux becomes almost zero at 500 kPa. Because vacuum is being applied on the other side of the membrane, the liquid does not break through. Evaporation can then take place instead of the liquid coming through.

Chau et al. provides examples where one pure organic solvent is obtained from a binary mixture of two organic solvents when an organic solvent reverse osmosis process is applied which involves application of considerably high feed pressure on the feed mixture while the downstream is at 1 atmosphere. (See, e.g., J. Chau et al., Organic Solvent Mixture Separation during Reverse Osmosis and Nanofiltration by a Perfluorodioxole Copolymer Membrane, J. Membrane Science, 618, 118663 (2021)). The process of vacuum membrane distillation amounts to the application of a driving force on any solvent species that is equivalent to about 300-400 atm pressure on the feed mixture in organic solvent reverse osmosis (See, e.g., FIG. 9 in Greenlaw et al., Dependence of diffusive permeation rates on upstream and downstream pressures I. Single Component Permeant, J. of Membrane Science, 2, 141-151 (1977)). Further, larger molecular weight solvent species have almost zero vapor pressure; as such, their partial pressure-based driving forces are much smaller, while the free volume dimensions in CMS 7 and AF2400 copolymer membranes will generally reject those larger dimension molecules. Hence the performance discussed herein is excellent with complete blockages of larger species in the C7-C30 mixture.

The experimental results shown in FIGS. 2-5 convincingly demonstrate that ultrapurification of IPA to make it useful for chip cleaning in semiconductor industry applications is possible with perfluoropolymer membranes, such as CMS-7, AF 2400, CMS-3, and/or AF 1600. A vacuum membrane distillation process can be employed and such membranes can be in flat or hollow fiber or other suitable forms. The hollow fiber form can enable packing of large surface areas per unit device volume and can be useful for larger-scale commercial use. In some embodiments, the outside surface area of the hollow fiber can include a thin coating of CMS-7, and the vacuum can be applied through the bore of the hollow fiber. It is also possible to reverse the configuration with the coating of CMS-7 introduced in the hollow fiber internal diameter and the vacuum applied outside. The microporous hollow fiber substrate to be used for coating of CMS-7 can be made of the following solvent-resistant materials: polyetheretherketone (PEEK) and related polymers; polyacrylonitrile; polytetrafluoroethylene; cross-linked polyetherimide, polyimide; polyamide; polypropylene; polymethylpentene; polyethylene; polyphenylene sulfide; regenerated cellulose, perfluoroalkoxy (PFA) polymer; or the like.

The same membrane can be used to engage in ultrapurification of organic solvents, such as, e.g., toluene, ethanol, methanol, butanol, or the like, using the membrane distillation system discussed herein. In some embodiments, a sweep gas membrane distillation can be used with a sweep gas instead of a vacuum on the permeate side and then condense isopropyl alcohol in a condenser. The treatment temperature needs to be adjusted depending on the solvent. One of the primary benefits of the exemplary system and method is a significantly substantial reduction in the semi-volatile and nonvolatile impurities in the organic solvent being treated.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A method of purifying an organic solvent, the method comprising:
introducing an organic solvent into a membrane distillation system, the membrane distillation system including a perfluorodioxole membrane; and
performing a distillation technique with the membrane distillation system using the perfluorodioxole membrane to treat and purify the organic solvent.

2. The method of claim 1, wherein the perfluorodioxole membrane has a dioxole content of about 65% to about 87%.

3. The method of claim 1, wherein the perfluorodioxole membrane comprises a perfluoropolymer of perfluoro-2,2-dimethyl-1,3-dioxole copolymerized with tetrafluoroethylene membrane.

4. The method of claim 1, wherein the perfluorodioxole membrane comprises a HYFLON® AD perfluoropolymer or a CYTOP® AD perfluoropolymer.

5. The method of claim 3, wherein the distillation technique comprises a vacuum membrane distillation process.

6. The method of claim 3, wherein the distillation technique comprises a sweep gas membrane distillation process.

7. The method of claim 3, wherein the distillation technique comprises an air gap membrane distillation process.

8. The method of claim 3, wherein the distillation technique comprises a direct contact membrane distillation process.

9. The method of claim 1, wherein the organic solvent is isopropyl alcohol.

10. The method of claim 1, wherein the organic solvent is isopropyl alcohol, toluene, ethanol, methanol, or butanol.

11. The method of claim 1, wherein treating and purifying the organic solvent results in the organic solvent being free from all non-volatile contaminants.

12. The method of claim 1, wherein the perfluorodioxole membrane is a perfluorodioxole copolymer membrane.

13. The method of claim 1, wherein the perfluorodioxole membrane includes a pore size between about 1 nanometer and about a sub-nanometer.

14. A system for purifying an organic solvent, the system comprising:
a feed reservoir including an organic solvent;
a perfluorodioxole membrane fluidically connected to the feed reservoir; and
a pump configured to pump the organic solvent from the feed reservoir to and over the perfluorodioxole membrane;
wherein passage of some of the organic solvent through the perfluorodioxole membrane performs a distillation technique to treat and purify the organic solvent.

15. The system of claim 14, wherein the perfluorodioxole membrane includes a first side exposed to the organic solvent pumped to the perfluorodioxole membrane by the pump.

16. The system of claim 14, wherein the perfluorodioxole membrane includes a second side opposing the first side, and the second side is exposed to one or more vacuum levels.

17. The system of claim 16, comprising a vacuum pump fluidically connected to the second side of the perfluorodioxole membrane to expose the second side to the one or more vacuum levels.

18. The system of claim 16, comprising a cold trap fluidically connected to the second side of the perfluorodioxole membrane to collect condensate from the perfluorodioxole membrane.

19. The system of claim 14, comprising a temperature probe capable of measuring a temperature of the organic solvent prior to entry of the organic solvent into the perfluorodioxole membrane.

20. The system of claim 14, wherein the perfluorodioxole membrane comprises a perfluoropolymer of perfluoro-2,2-dimethyl-1,3-dioxole copolymerized with tetrafluoroethylene membrane.

21. The system of claim 14, wherein the distillation technique comprises at least one of a vacuum membrane distillation process, a sweep gas membrane distillation process, an air gap membrane distillation process, or a direct contact membrane distillation process.

22. The system of claim 14, wherein the organic solvent is isopropyl alcohol, toluene, ethanol, methanol, or butanol.

23. The system of claim 14, wherein treating and purifying the organic solvent results in the organic solvent being free from all non-volatile contaminants.
